(19) United States
(12) Patent Application Publication
(10) Pub. No.: US 2025/0177699 A1
(43) Pub. Date: Jun. 5, 2025

(54) MECHANIZED ACTUATION OF CATHETERS

(71) Applicant: EDWARDS LIFESCIENCES CORPORATION, Irvine, CA (US)

(72) Inventors: Nikolai Brent Poulsen, Tustin, CA (US); Christopher Aguayo, Irvine, CA (US)

(21) Appl. No.: 19/043,461

(22) Filed: Feb. 1, 2025

Related U.S. Application Data
(63) Continuation of application No. PCT/US2023/029356, filed on Aug. 2, 2023.
(60) Provisional application No. 63/395,159, filed on Aug. 4, 2022.

(51) Int. Cl. A61M 25/01; A61F 2/24

(52) U.S. Cl. CPC A61M 25/0113; A61F 2/2427; A61M 2205/332; A61M 2205/502

(57) ABSTRACT

A medical system comprises an electronically controlled actuator system and a catheter system for providing access within a body cavity. The electronically controlled actuator system includes a plurality of carriages mounted with a frame and movable along a longitudinal axis with respect to the frame, and a carriage drive system configured to adjust a position of each of the carriages. The catheter system includes a distal end, a proximal end, a plurality of lumens extending between the distal end and the proximal end, and a plurality of adapters. Each of the adapters can be coupled to a corresponding carriage and attach to a lumen such that adjusting the position of the corresponding carriage actuates the attached lumen. The actuator system has a control system with executable instructions to control the plurality of carriages and therefore the lumens of the catheter system.

### CROSS-REFERENCE TO RELATED APPLICATIONS

This application a continuation of International PCT Application No. PCT/US2023/029356 filed Aug. 2, 2023, which claims the benefit of U.S. Provisional Application No. 63/395,159, filed Aug. 4, 2022, the entire content of which is hereby incorporated herein by reference.

### FIELD

Embodiments disclosed herein relate generally to catheter systems for delivery of a prosthesis and control systems thereof.

### BACKGROUND

Human heart valves, which include the aortic, pulmonary, mitral and tricuspid valves, function as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally procedures and even non-invasive percutaneous procedures. Such replacement valves often include a tissue-based valve body that is connected to an expandable frame that is then delivered to the native valve's annulus.

Development of prostheses including, but not limited to, replacement heart valves that can be compressed for delivery and then controllably expanded for precise implantation has proven to be challenging. An additional challenge relates to the ability of such prostheses to be secured relative to intralumenal tissue, e.g., tissue within any body lumen or cavity, in an atraumatic manner.

Delivering a prosthesis to a desired location in the human body, for example delivering a replacement heart valve to the mitral valve, can also be challenging. Obtaining access to perform procedures in the heart or in other anatomical locations may require delivery of devices percutaneously through tortuous vasculature or through open or semi-open surgical procedures. Catheter-based implantation of a prosthesis can particularly challenging when numerous catheter shafts and actuation mechanisms are operated by the clinician during the procedure. Accordingly, there is a need to simplify the procedure and improve the clinician's ability to precisely control the movement of catheters through the vasculature and the deployment of the prosthesis at the desired location.

### SUMMARY

A medical system comprises an electronically controlled actuator system and a catheter system for providing access within a body cavity. The electronically controlled actuator system can include a frame extending along a longitudinal axis, a plurality of carriages mounted with the frame and movable along the longitudinal axis with respect to the frame, and a carriage drive system configured to adjust a position of each of the carriages along the longitudinal axis based on at least one control signal. Each of the plurality of carriages can include a nest coupled thereon, configured to rotate about the longitudinal axis. The catheter system can include a distal end, a proximal end, a plurality of lumens extending between the distal end and the proximal end, and a plurality of adapters. Each of the adapters can attach to a lumen of the plurality of lumens. Preferably, each of the plurality of adapters couples to a corresponding carriage of the plurality of carriages such that adjusting the position of the corresponding carriage along the longitudinal axis actuates the attached lumen.

According to another aspect, the actuator system has a control system with at least one computer-readable memory having stored thereon executable instructions and one or more processors in communication with the at least one computer-readable memory to execute the instructions to cause the system to at least: receive an input signal and generate the at least one control signal based on the input signal. According to another aspect, the actuator system has a user interface for providing the input signal. According to another aspect, the user interface is located remotely from the actuator system and in wired or wireless communication with the control system. According to another aspect, the plurality of lumens of the catheter system include an outer sheath assembly, a mid-shaft assembly, a rail assembly, and an inner shaft assembly.

According to another aspect, a first load cell measures a force applied to a first lumen of the plurality of lumens by a first carriage of the plurality of carriages. According to another aspect, first load cell is mounted on the first carriage and contacts a first adapter of the plurality of adapters when coupled with the first load cell. According to another aspect, a user interface displays the force measured by the first load cell relative a permitted force limit associated with the first lumen. According to another aspect, the user interface displays an available range of motion of the first lumen based on the force measured by the first load cell and the permitted force limit. According to another aspect, executing the instructions further causes the system to: receive a force signal from the first load cell and generate the at least one control signal further based on a comparison of the force signal with the permitted force limit. The at least one force signal prevents exceeding the one or more permitted force limits.

According to another aspect, the at least one control signal adjusts the positions of at least two of the plurality of carriages along the longitudinal axis as a first group. According to another aspect, each of the carriages of the plurality of carriages includes a receiving slot configured to receive one of the plurality of adapters therein. According to another aspect, each of the adapters includes one or more lateral protrusions that extend perpendicularly to the longitudinal axis of the frame and each of the receiving slots includes one or more upwardly projected configured to engage with the one or more lateral protrusions. According to another aspect, the proximal end of the catheter system includes a handle shell including one or more shell members. According to another aspect, the plurality of adapters protruding laterally outwardly from within the handle shell. According to another aspect, the handle shell is removable. According to another aspect, the frame is rotatable on a rotation axis aligned parallel or generally along the longitudinal axis. According to another aspect, a first pivot mount connects with a first end of the frame and a second pivot mount connects with a second end of the frame, the first and second pivot mounts aligned along the rotation axis. According to another aspect, the plurality of lumens are attached with the plurality of adapters by respective guidewires. According to another aspect, a base or table includes an upper surface to support the frame. According to another aspect, a patient bed supports the base or table.

According to another aspect, the frame comprises a pair of rails extending parallel with the longitudinal axis and the plurality of carriages are mounted on the pair of rails. According to another aspect, the carriage drive system comprises a motor for each of the plurality of carriages. According to another aspect, the carriage drive system comprises a rack attached with the frame and each of the motors includes a pinion gear meshed with the rack. According to another aspect, each of the plurality of carriages includes a limit switch or proximity switch for homing the carriages relative to the frame. According to another aspect, the catheter system can be any catheter system from a set of catheter systems including a plurality of universal adapters. According to another aspect, the plurality of carriages are repositionable to receive any catheter system from the set of catheter systems in an initial pre-programmed spacing configuration. According to another aspect, the at least one control signal is further based on an autonomous or semi-autonomous control algorithm.

In another aspect, a method includes positioning an electronically controlled actuator system relative to a patient. The electronically controlled actuator system has a frame extending along a longitudinal axis, a carriage mounted with the frame is movable along the longitudinal axis with respect to the frame, and a carriage drive system adjusts a position of the carriage along the longitudinal axis. The method includes making an incision to access a vasculature of the patient. The method includes inserting a distal end of a catheter system into the vasculature of the patient through the incision and advancing the distal end through the vasculature into or adjacent to an anatomical region of the patient. The method includes aligning a proximal end of the catheter system relative to the electronically controlled actuator system. The method includes coupling an adapter of the catheter system with the carriage. The method includes receiving at least one control signal at a control system of the electronically controlled actuator system. The method includes adjusting a position of the carriage along the longitudinal axis based on at least one control signal and thereby actuating a lumen of the catheter system.

According to another aspect, the input signal is from a user interface and generates the at least one control signal based on the input signal. According to another aspect the method includes receiving a force signal from a load cell and generating the at least one control signal further based on the force signal. According to another aspect the method includes calculating an available range of motion of the lumen based on the force signal measured by the first load cell and a permitted force limit of the lumen. According to another aspect the method includes adjusting the position of the carriage to an initial, pre-programmed location before coupling the adapter of the catheter system with the carriage. According to another aspect, the catheter system comprises a plurality of lumens and a plurality of corresponding adapters, and the electronically controlled actuator system includes a plurality of carriages.

The foregoing summary is illustrative only and is not intended to be limiting. Other aspects, features, and advantages of the systems, devices, and methods and/or other subject matter described in this application will become apparent in the teachings set forth below. The summary is provided to introduce a selection of some of the concepts of this disclosure. The summary is not intended to identify key or essential features of any subject matter described herein.

### BRIEF DESCRIPTION OF THE DRAWINGS

Various examples are depicted in the accompanying drawings for illustrative purposes, and should in no way be interpreted as limiting the scope of the examples. Various features of different disclosed examples can be combined to form additional examples, which are part of this disclosure.

FIG. 1 schematically illustrates an electronically controlled actuator including a plurality of movable carriages in use with a catheter system;

FIG. 2 illustrates a perspective view of the electronically controlled actuator shown in FIG. 1;

FIG. 3 illustrates a partial perspective view of another electronically controlled actuator;

FIGS. 4-5 illustrate partial perspective views of another electronically controlled actuator;

FIG. 6 illustrates an adapter for connecting a lumen of a catheter system with a carriage of an electronically controlled actuator;

FIGS. 7-8 illustrate partial perspective views of another electronically controlled actuator;

FIG. 9 illustrates another electronically controlled actuator;

FIGS. 10-11 schematically illustrate an implementation to cause translational and rotational movements of a lumen using two motors respectively;

FIG. 12 schematically illustrate another implementation to cause translational and rotational movements of a lumen using two motors respectively;

FIG. 13 schematically illustrates an implementation to convert translational movement to rotational movement;

FIG. 14 schematically illustrates an implementation to cause translational and rotational movements of a lumen using one motor;

FIG. 15 is a control method for an electronically controlled actuator.

### DETAILED DESCRIPTION

The various features and advantages of the systems, devices, and methods of the technology described herein will become more fully apparent from the following description of the examples illustrated in the figures. These examples are intended to illustrate the principles of this disclosure, and this disclosure should not be limited to merely the illustrated examples. The features of the illustrated examples can be modified, combined, removed, and/or substituted as will be apparent to those of ordinary skill in the art upon consideration of the principles disclosed herein.

Transcatheter repair and native anatomy replacement procedures can require the use of complex catheter systems, the dexterity to make fine adjustments to catheter controls, and expert interpretation of fluoroscopy and/or echo imaging modalities to properly treat patients. As the demand of additional functionality is placed upon catheter technologies, new failure modes are likely to occur. Mechanized systems are far more stable than the human hand and can make more precise and repeatable movements. Imaging software is currently in development to use the various imaging modalities to create a better representation of the interactions between the catheter and patient anatomy. With these three points in mind, a system capable of actuating catheter systems would lead to a safer, more repeatable, and more precise procedure.

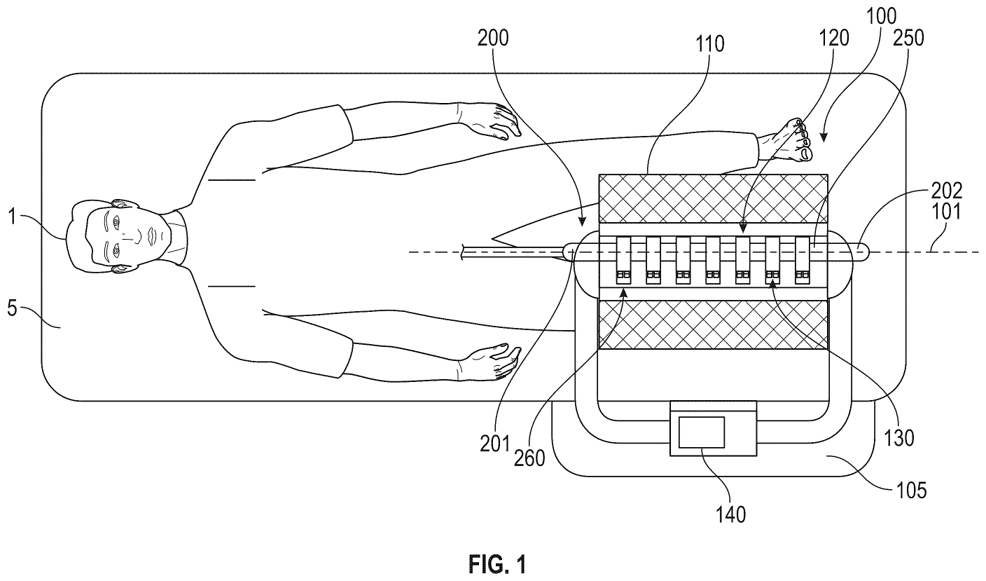

FIG. 1 illustrates medical system comprising an electronically controlled actuator 100 and a catheter system 200 in use with a patient 1 resting on a patient support 5. The electronically controlled actuator or system 100 can include a frame 110. The frame 110 can extend along a longitudinal axis 101. The frame 110 can be supported by a base 105. The base 105 can rest on a floor, on the patient support 5 or on another structure. The base 105 can be mobile and/or fixable in position relative to the patient support 5. The base 105 can position the frame 110 relative to the patient 1 for proper positioning of the catheter system 200.

The frame 110 can support a plurality of carriages 120 mounted with the frame 110. The carriages 120 can be movable with respect to the frame. For example, the carriages 120 can be movable along the frame 110 (e.g., the longitudinal axis 101). The carriages 120 can be aligned along the longitudinal axis 101. Each of the carriages 120 can include a slot or other coupling feature configured to hold a lumen of a catheter. Each of the carriages can include a load cell for measuring a force related (e.g., applied) to each carriage.

A carriage drive system 130 can include actuators, motors and/or other mechanisms to control positions of each of the carriages 120 within the frame 110. Each of the carriages 120 can be independently movable or movable in sub-groups (e.g., pairs) relative to any of the other carriages by the drive system 130. The drive system 130 can include position sensors for tracking the carriages 120 along the frame 110 (e.g., along longitudinal axis 101). The drive system 130 can include limit switches and/or proximity switches for homing the carriages 120 at positions along the frame 110 (e.g., along longitudinal axis 101) and/or relative to other carriages.

The carriage drive system 130 can include a control system for adjusting the positions of the carriages 120. The control system can include at least one computer-readable memory having stored thereon executable instructions and one or more processors in communication with the at least one computer-readable memory to execute the instructions. The executed instructions can receive an input signal and generate at least one control signal based on the input signal. The input signal can include a set of instructions on adjusting the positions of one or more of the carriages 120. The input signal can be sent manually, such as through a user interface 140 including one or more controls, or generated by an autonomous or semi-autonomous control algorithm. The user interface 140 can be local (e.g., on the base 105) or remote from the frame 110. The user interface 140 can be in wired or wireless communication with the control system.

The catheter system 200 can provide access to a work site within a body cavity for one or more instruments. Various types of catheter systems 200 are contemplated herein. In one example, the catheter system 200 includes a deployable replacement heart valve, such as the delivery systems described in U.S. Pat. Pub. No. 2019/0008640, the entirety of which is hereby incorporated by reference. The catheter system 200 can include a distal end (not shown) which may be attached to a distal end 201 of a handle and a proximal end 202. The distal end can be insertable within a vasculature of the patient 1 to access a remote work site such as the patient's heart through a transfemoral approach.

The catheter system 200 can include a plurality of lumens 250 extending between the distal end and the proximal end. The term “lumen” herein may refer to a shaft or tube or other physical structure having a lumen therein. The lumens 250 can be encased or nested together, meaning smaller lumens can be encased within larger lumens, with all lumens having about the same length. In this way, the plurality of lumens 250 can be encased or nested together as one assembly, allowing a lumen to move longitudinally relative to the other lumens. When each of the lumens 250 is held by one of the carriages 120 of the system 100, each the lumens 250 can translate longitudinally independently. The lumens 250 can include an outer sheath assembly, a mid-shaft assembly, a rail assembly, and an inner shaft assembly. The lumens 250 can be actuated to deploy a prosthetic at the work site. The proximal end 202 of the catheter system 200 can include a handle and/or one or more control features for the lumens 250. The proximal end 202 of the catheter system 200 can include a handle shell including one or more shell members. The shell members can fully or partially enclose the control features. The shell members can be fully or partially removable to access the control features. The control features can include a plurality of adapters 260. Each of the adapters 260 can be attached to a lumen of the plurality of lumens 250, such as through a guidewire. The adapters 260 can be coupled with a corresponding one of the carriages 120. In one example, the adapters 260 can be received within a slot within corresponding carriages 120. In another example, the carriages 120 can include clamps, protrusions that engage with (e.g., insert within) the respective adapters, or other coupling mechanisms. Accordingly, adjusting the positions of the carriages 120 along the frame 110 (e.g., along the axis 101) actuates the attached lumens 250.

In certain implementations, an operator can interact with the system 100 to control the catheter system 200 through the user interface 140. The user interface 140 can include a selection interface for selection of one or more lumens 250 (e.g., individually or as sub-groups). The user interface 140 can include controls for adjusting the positions of the selected one or more lumens 250 (e.g., controlled adjustments or manual adjustment).

The carriage drive system 130 can move the carriages 120 into one or more pre-programmed configurations. The pre-programmed configurations can correspond to initial and/or other operative positions of one or more control features of the catheter system 200. The initial configuration can be used to accommodate various different catheter system designs or different medical procedures that may use different initial spacings for the adapters 260. The carriage drive system 130 can move the carriages 120 into position based on an input from an operator at the user interface 140. The user interface 140 can include a selection menu of various pre-programmed configurations for the system 100. The selections can be based on the type of catheter system and/or the medical procedure.

The system 100 can include automated controls for executing one or more steps of a medical procedure using the catheter system 200. The steps of a medical procedure and the required movements of the carriages 120 and lumens 250 corresponding thereto can be pre-programmed into the system 100. The user interface 140 can include selections corresponding to each of the steps of the medical procedure. The operator can execute the steps of the medical procedure based on selection of the appropriate procedure (e.g., step). The selection (e.g., execution) of the steps can automatically move the carriages 120 according to a pre-programmed movement and/or location. For certain catheter systems, releasing tension or compression on a first lumen for a first movement before proceeding with applying tension or compression of a second lumen for a second movement can be critical to prevent failures in the catheter system. Accordingly, the system 100 can automatically neutralize or back drive lumens during or between steps of the medical procedure.

The automated procedural steps executed by the system 100 and catheter system 200 can be based on detected positions of the catheter system 200. Imaging modalities can be used to provide images of anatomical structures and the catheter system 200 during the medical procedure that are pertinent to success. Advancements in imaging modalities continue to improve resolution of such images. Software, including machine learning techniques, can be used to interpret these images and recognize with high confidence when the catheter system 200 is in an appropriate position for a procedural step or when the catheter system 200 is not in an appropriate position for the procedural step. This determination can be linked with the system 100 and/or provided to the operator. As such, the operator or system 100 has opportunity to adjust the catheter system 200 manually or approve automatic adjustments. When conducted by the system 100, the process can provide a high level of precision and speed to the procedure. Desirably, the system 100 can be fully autonomous or allow the operator to accept recommended procedural moves.

The system 100 can measure force feedback signals. The force feedback signal can be generated from sensors such as load cells mounted on the carriages 120, based on the current of the motors of the carriage drive system 130, and/or other means. The carriage drive system 130 can move the carriages 120 based on the force feedback signals in a manner that prevents exceeding one or more force thresholds of the system (e.g., thresholds of each particular lumen). Optionally, the user interface 140 can include warnings that the system has prevented/exceeded any such threshold. Optionally, the user interface 140 can provide for a manual override of one or more safety thresholds. In one example, a load cell measures a force applied to a lumen of the plurality of lumens 250 by a carriage of the plurality of carriages 120. The load cell can be mounted on the first carriage and contact an adapter of the plurality of adapters 260 when coupled with the carriage. The user interface 140 can display the force measured by the load cell and/or compare the force with a permitted force limit associated with the lumen. The user interface 140 can display an available range of motion of the lumen based on the force measured by the load cell and the permitted force limit. Optionally, the permitted force limit can be exceeded based on an operator input. Having the ability to measure forces live, move multiple lumens at once, and have full awareness of where each lumen is relative to another may advantageously allow for added features that are difficult to implement with a traditional handle design. For example, these features can be used for mitigation of safety risks and procedural maneuvering.

The carriage drive system 130 can move the carriages 120 based on limits to the rate of translation for any of the lumens/carriages. Each lumen can have a target moving rate or range of rate (upper and lower rates). The target moving rates can be varied depending on the step of the medical procedure being performed. The translations rates and/or the force thresholds can be based on failure testing of the catheters systems. The translation rates and/or the force thresholds can set to overcome any internal friction of the catheter system without overshooting a desired location. Irregularities, such as an incomplete maneuver, or malfunctions, such as binding of a lumen, in the catheter system during any step of the medical procedure can be detected using the force feedback signal. The range of forces required to move any carriage for a given configuration of the catheter system can be known based on models and/or testing. Accordingly, deviation from the expected ranges can be automatically recognized. A warning can be provided to the operator through the user interface.

Fluid pressure sensors can also be implemented in the electronically controlled actuator system to provide information to the operator or control system during a procedure. Connecting tubing to the flush ports that may be ubiquitous on a catheter would allow access to pressures directly surrounding the catheter inside of the body. One example use case can be for heart valve replacement. Maneuvers that pin leaflets of a valve may result in pressure drops which can be harmful to a patient. The fluid pressure sensors can provide feedback to the operator or automatically to the motors to reverse or cease the catheter movement in a time-sensitive manner.

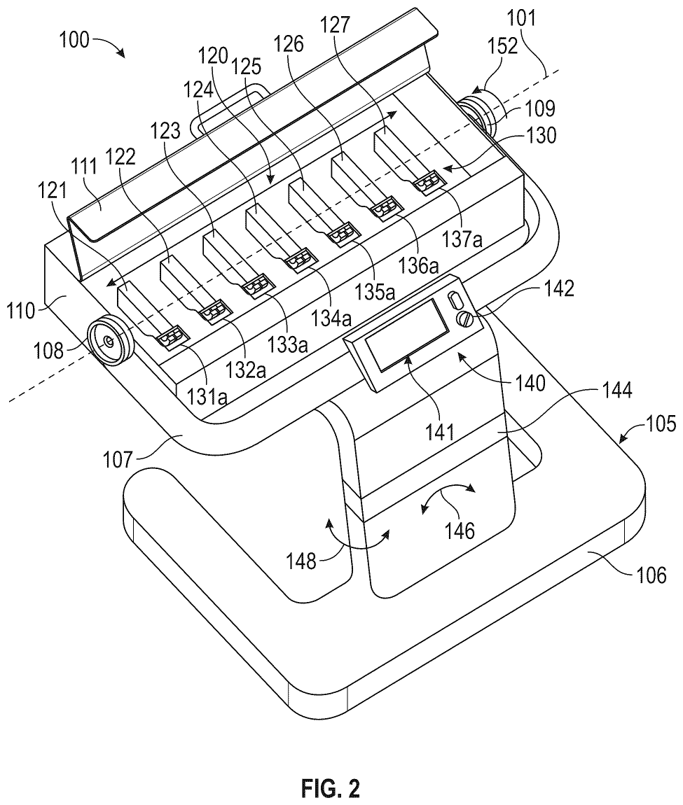

FIG. 2 shows further detail of the system 100. The system 100 can include the frame 110 supported by the base 105. The base 105 can include a lower support 106. The lower support 106 can be configured to rest on a bed or a floor. The base 105 can include an upper support 107 extending vertically upwardly from the lower support 106. A joint 144 or pivot mechanism or other rotational mechanism can be disposed between the lower support 106 and the upper support 107. The joint 144 can be a universal joint or the like, allowing the upper support 107 to turn, pivot or bend in a direction 146 so that the catheter system 200 can tilt up and down. The joint 144 can also allow the upper support 107 to turn in a direction 148 so that the catheter system 200 can move laterally. As such, the distal end of the catheter system 200 can be moved to accurately aim the work site within a body cavity (e.g., accommodate angle into a patient) to perform a medical treatment or diagnostic procedure (e.g., the medical procedure mentioned above) without moving the base 105. After the adjustments, the joint 144 can be locked through a locking mechanism so that the catheter system 200 does not move inadvertently during the procedure. The upper support 107 can include first and second pivot controls 108, 109. The pivot controls 108, 109 can be pivotably coupled with the frame 110. The pivot controls 108, 109 can be aligned along the longitudinal axis 101. The pivot controls 108, 109 rotated along a direction 152 to allow rotation of the frame 110 about the longitudinal axis 101 to control a rotational position of the catheter system 200 during a medical procedure. The upper support 107 can include a mounting position of the user interface 140 (or a portion thereof). The user interface 140 can include a display screen 141 and/or an input/control device 142.

The frame 110 can include a lid 111. The lid 111 can enclose a cavity containing the carriages 120. The carriages 120 can include a plurality of carriages, e.g., first through seventh carriages 121-127 as shown in FIG. 2. One or more of the carriages 120 can include a motor controller, as described above, e.g., first to seventh motor controller 131a-137a. The motor controllers 131a-137a can be mounted on the carriages 121-127, respectively, and operable to move the carriages along the longitudinal axis 101. One or more of the carriages 120 can include a load cell, as described above. The load cells can be mounted on the carriages 121-127, respectively, and operable to measure forces applied by the carriages to the catheter system 200.

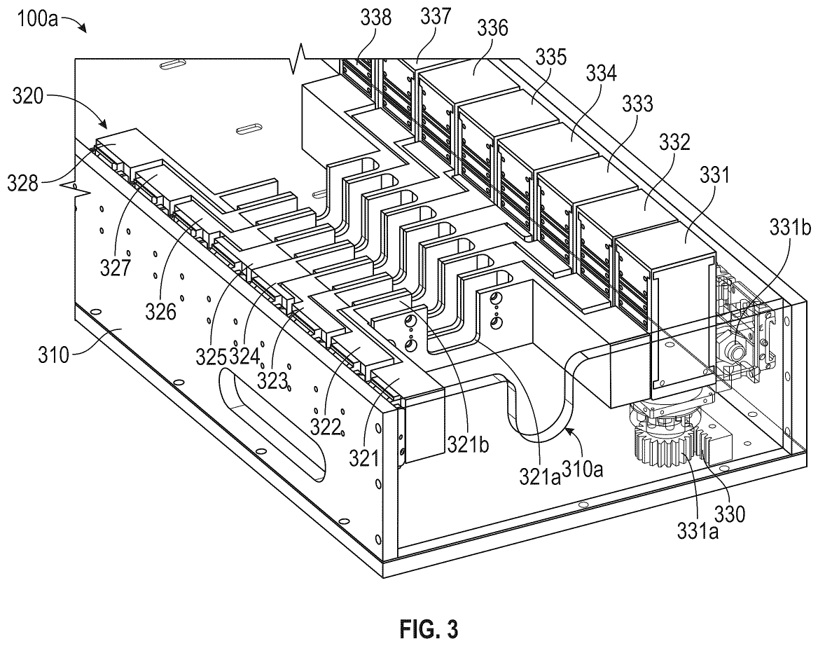

FIG. 3 shows a partial perspective view of an electronically controlled actuator or system 100a as one implementation of the system 100. The system 100a can include a frame 310. A front wall of the frame 310 can include a cutout 310a that receives a portion of the catheter system 200 when assembled therewith. The system 100a can include the carriages 320. The carriages 320, like the carriages 120 illustrated in FIG. 1, can be movable along the frame 310. The carriages 320 can include a plurality of carriages, e.g., first through eighth carriages 321-328. As can be seen in FIG. 3, outer prongs of the carriages (e.g., 321, 322, 323, 326, 327, 328) can be shaped (e.g., U-shaped) and configured to nest within adjacent carriages. Each carriage 320 can include a slot for receiving an adapter of the catheter system 200. For example, the first carriage 321 has a slot defined between side walls 321a, 321b to accept an adapter. Each carriage 320 can include a motor of a carriage drive system, such as the first through eighth motors 331-338. Each motor can include a pinion gear (such as gear 331a) that can mesh with a rack 330 that extends along the frame 310 in a direction of movement. The carriages 320 can be moved along the rack 330 by actuation of the motors. One or more of the carriages 320 can include a limit switch or proximity switch 331b for homing the carriages relative to the frame and/or other carriages. Each carriage 320 can include one or more mounts for fixation of a port, such as a flush port. Alternatively, or in addition to providing linear movement, one or more carriages can be included that provide different types of movement. In one example, a carriage can include a rotational driver, as will be further described subsequently. The rotational driver can be used to deploy and/or stow an elongate member, such as a suture or tether.

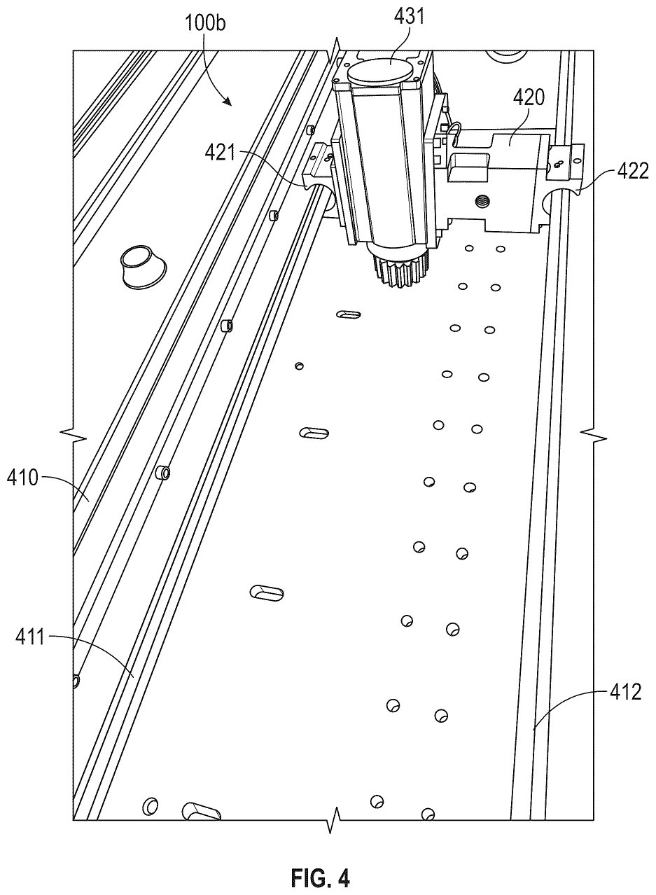

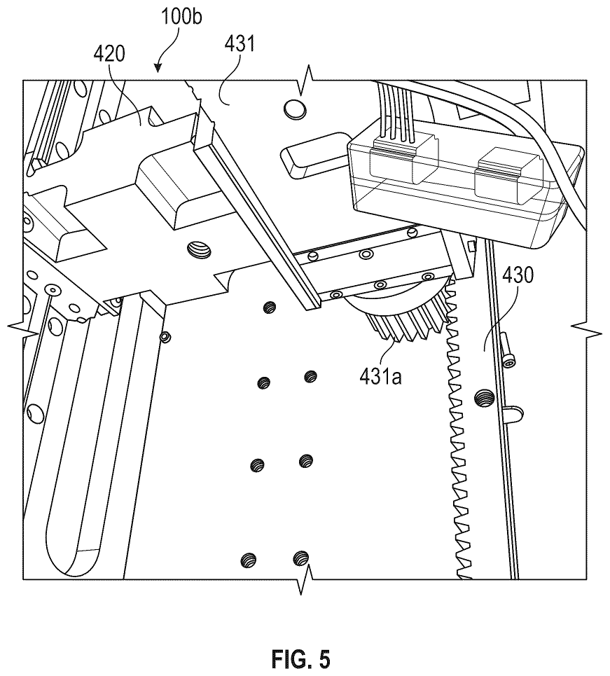

FIG. 4-5 show partial perspective views of an electronically controlled actuator 100b as another implementation of the system 100. The system 100b can include a frame 410 that includes first and second side rails 411, 412. A carriage 420 can be mounted on the rails 411, 412 by slots 421, 422, respectively, and movable there along. A motor 431 can be coupled with the carriage 420. The motor 431 can engage with a rack 430 through a pinion gear 431a in the manner described above with system 100a.

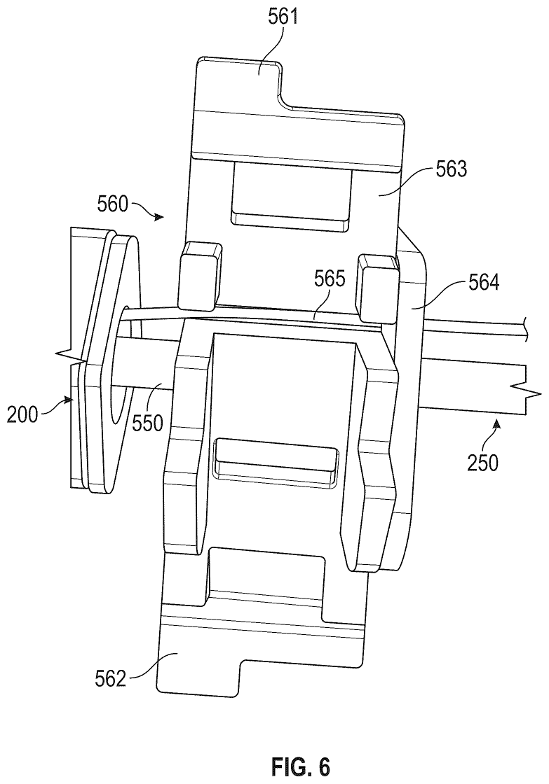

FIG. 6 shows an example adapter 560 like the adapters 260 of the catheter system 200 described with respect to FIG. 1. The adapter 560 can include a body 563 that can be insertable within a carriage (e.g., within a slot thereof). The slot of the carriage can be open in the vertical direction (e.g., as shown in FIG. 3) and the adapter 560 can be inserted into the slot along the vertical direction. The adapter 560 can include protrusions 561, 562 that extend from the body. The adapter 560 can be coupled with (e.g., attached to) a lumen 250 of the catheter system 200 through a guidewire 565. The guidewire 565 can be attached with the body 563 through a slot and/or a plate 564. The body 563 can include an aperture through which lumens 550 of the catheter system 200 can pass. The lumens 550 can include a plurality of lumens encased or nested together, as described above. Among the plurality of lumens 550, the lumen 250 may be the only one that is attached to the adapter 560. The protrusions 561, 562 can engage with opposing walls of the carriage to allow the system 100 to actuate the lumen 250 of the catheter system. As such, moving the carriage engaged with the adapter 560 moves the lumen 250 attached to the adapter 560. The shape of the adapter 560 can vary as long as there is material sufficient to mechanically fixate the adapter relative to the carriage. The shape of the adapter 560 can be standardized for catheter systems to limit the number of unique nest designs of the carriages. The catheter system 200 may include a handle shell at the proximal end. The handle shell may be permanent and allow for fixation of the adapters into the actuator, platform or system 100, 200 or the handle shell could be a temporary packaging that maintains orientation of lumens and adapters and is removed when the catheter system is placed into the actuator, platform or system 100, 200. In this case, the adapter 560 can reside within the handle shell to couple to the lumen 250, and the handle shell can include apertures allowing the protrusion 561, 562 to penetrate through to engage with the carriage. In other embodiments, the catheter system 200 has no handle shell.

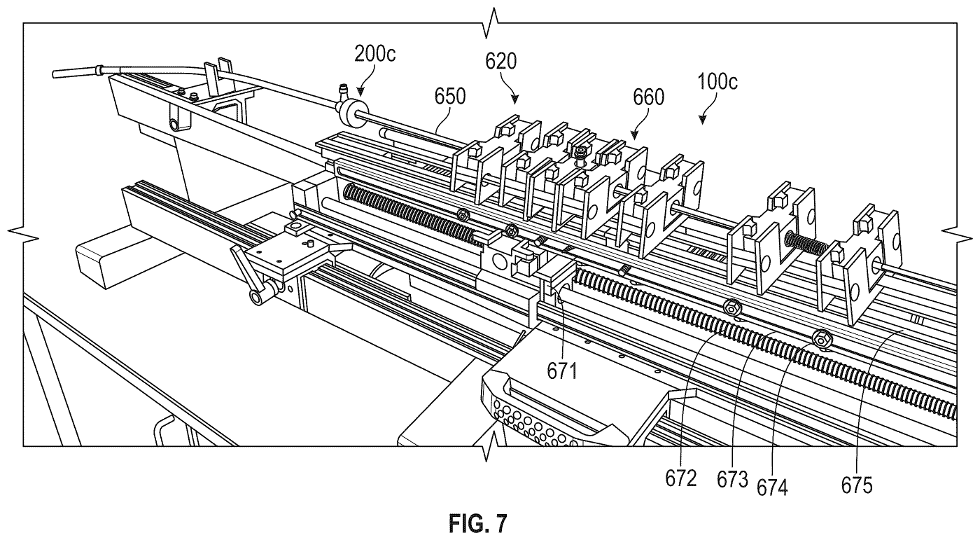

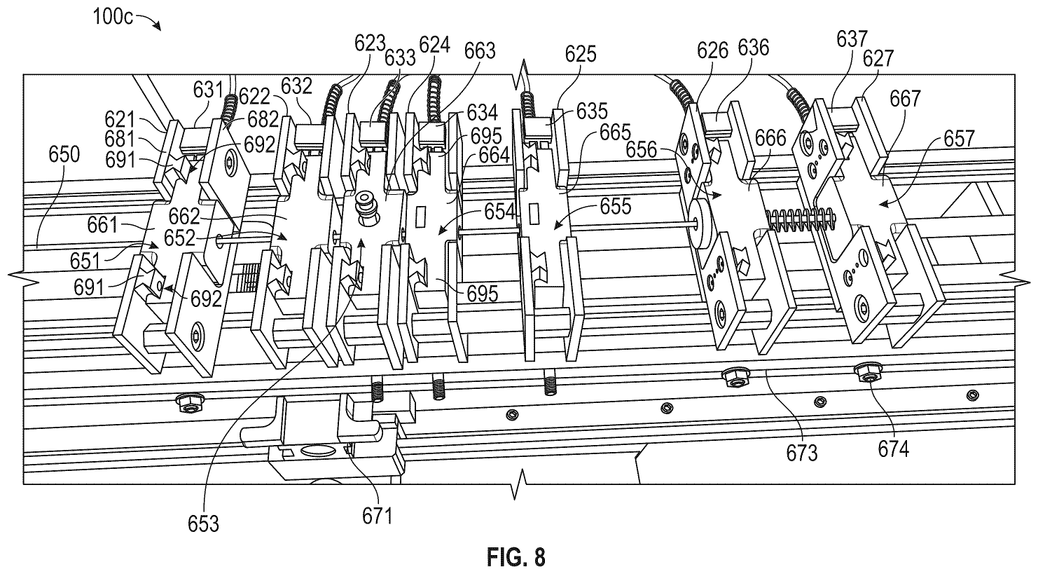

FIGS. 7-8 show an electronically controlled actuator or system 100c as one implementation of the system 100 and a catheter system 200c. The system 100c can include a plurality of carriages 620, e.g., including first through seventh carriages 621-627. The carriages, like the carriages 120 shown in FIGS. 1-2, can be movable along a support rail 675. Each carriage 620 can include a slot (e.g., slots 651-657) for receiving a corresponding an adapter of a plurality of adapters 660 of the catheter system 200c. The adapters 660 can include first through seventh adapters 661-667. The adapters 661-667 can be received or nested within slots of the corresponding carriages 621-627 that can be open in the vertical direction. Lateral protrusions 695 of the adapters 660 can engage with the sides of the slots to allow the carriages to apply forces to the catheter system 200c through the adapters. Alternatively, the catheter system 200c can have a handle including a shell to provide a grip surface. The shell can be removed before the adapters 660 are coupled with the carriages 620. Alternatively, the shell is never included in the system 200c. The adapters 660 can each be coupled with a component of the catheter system 200c, such as with one of a plurality of control features or lumens 650. The plurality of lumens 650 can include an outer shaft, mid-shaft, rail assembly, primary flex, secondary flex, inner shaft and/or a guidewire lumen, as described in U.S. Pat. Pub. No. 2019/0008640.

The carriages 620 can be mounted on the support rail 675 and moveable along a longitudinal axis thereof. The carriages 620 can be mounted to a side rail 673 that extends along the support rail 675. A bolt and nut 674 can be used to selectively couple (e.g., attach) the carriages 620 with the side rail 673. The carriages 620 can be attached with the side rail 673 either singly or in groups. The carriages 620 can be moved along the support rail 675 by rotation of a worm gear 672 controlled by the motor control system. The side rail 673 can be attached with the worm gear 672 by a slide 671. Alternatively, each carriage 620 can include a motor of a carriage drive system. Each motor can include a pinion gear (not shown) that can mesh with a rack that extends in the direction of movement of the carriages. The carriages 620 can be moved along the rack by actuation of the motors.

Each carriage 620 can include a load cell, such as the load cells 631-637. The load cells 631-637 can form a “floating bridge” between front and back plates 681, 682 on each carriage. Each load cell can be attached to both the front and back plates 681, 682. The front and back plates 681, 682 can also form a slot that receives the respective adapters (e.g., the lateral protrusions thereof). Accordingly, tension and/or compression forces applied by the carriage to the catheter system through the carriage can be measured by the load cells 631-637. In some implementations, procedural movements are taken (e.g., by turning knobs) to return the load of specific lumens back to zero. This may be performed as a best guess at balancing load. With the load cells 631-637 implemented as described herein, one in each carriage, as shown in FIG. 8, returning load to zero can advantageously be done automatically and precisely. The front and/or back plates 681, 682 can include one or more projection rails 691. The projection rails 691 can be oriented vertically along a direction of insertion of the adapters. The adapters 660 can include alignment slots 692 that correspond to the size and position of the projection rails 691.

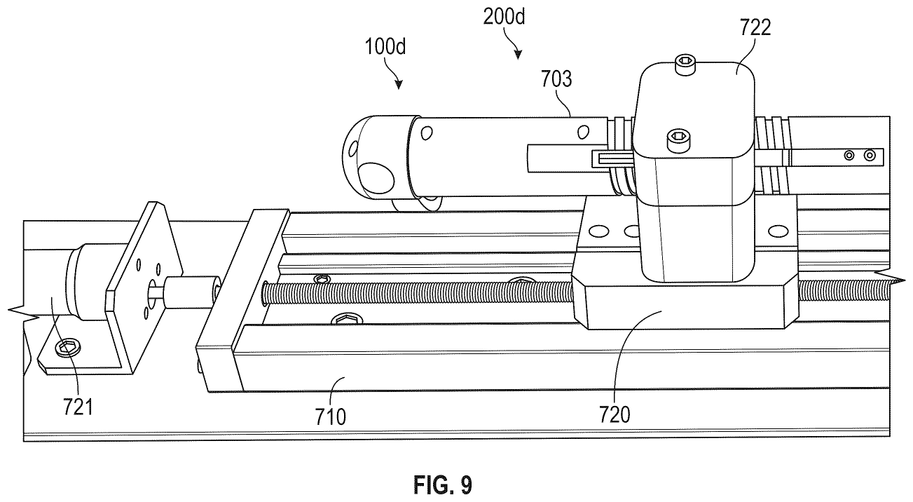

FIG. 9 shows an electronically controlled actuator or system 100d as another implementation of the system 100 and a handle 703 of a catheter system 200d. Most catheter designs rely on a slide or knob actuated by the user to move the lumen or lumens of the catheter. For the system 100d, the lumens can be actuated by a member or nest 722 connected to a carriage 720 that is driven by means alternative to manual manipulation (e.g., electromechanical, hydraulic, pneumatic). A lumen of the catheter system 200d can be attached to an adapter, such as the adapter 560 illustrated in FIG. 6, that has protrusions that stick out of shells of the catheter handle 703. The protrusions are clasped by the nesting component 722 that is on the carriage 720 driven by a motor 721. The shells of the handle 703 are not necessarily needed if there is sufficient support by the members driving the lumens although a basic handle shell may be desired for preparation and insertion of the device into the patient.

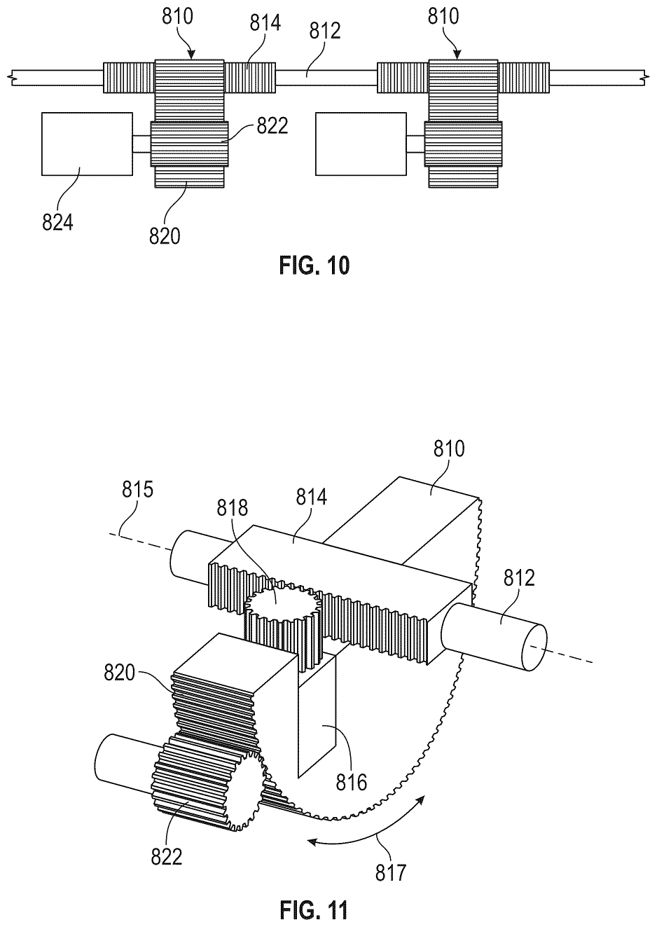

FIG. 10 illustrates a schematic front view of two nests 810 coupled with lumens 812. The two nests 810 may be part of a plurality of nests engaged with the lumens 812. Each of the nests 810 can be coupled with a carriage similar to the nest 722 and carriage 720 coupling shown FIG. 9, where the carriage 720 is part of the electronically controlled actuator 100d. The nests 810 can also be coupled to the carriages 120 of the electronically controlled actuator 100 illustrated in FIGS. 1 and 2, the carriages 320 of the electronically controlled actuator 100a illustrated in FIG. 3, the carriages 420 of the electronically controlled actuator 100b illustrated in FIGS. 4 and 5, or the carriages 620 of the electronically controlled actuator 200c illustrated in FIGS. 7 and 8. Each of the nests 810 shown in FIG. 10 is engaged with or to a rack 814, which is attached to an individual lumen of the plurality of lumens 812. The coupling of each respective nest 810 with the rack 814 can be through an adapter (e.g., the adapter 560 described above). In FIG. 10, it is also shown that each nest 810 has gear teeth 820 to engage a gear 822, which is actuated by a motor 824.

FIG. 11 illustrates a schematic perspective view of one of the nest-to-lumen engagements shown in FIG. 10. It can be seen that the rack 814 is attached to a lumen, which is one of the plurality of the lumens 812. The rack 814 is driven by a pinion gear 818 that is actuated by a motor 816. The rotation of the pinion gear 818 can cause the rack 814 to translate in a longitudinal axis 815 of the lumens 812. As described with respect to FIG. 1, this translational movement of the rack 814 attached to a lumen can be independent of the translational movement of other lumens. The implementation of the pinion gear 818 and the rack 814 to translationally move a lumen is an alternative to the pinion gear 331a and the rack 330 engagement shown in FIG. 3. Meanwhile, the gear 822 can be actuated by the motor 824 to cause the nest 810 to rotate in a rotational direction 817 when the teeth of the gear 824 engage with the teeth 820 of the nest 810. As constructed in FIG. 11, the nest 810 can rotate about the longitudinal axis 815, and thus also rotates the lumens 812. A respective lumen can be locked to a rotational position through a locking or breaking mechanism (not shown). As such, the implementation as shown in FIGS. 10 and 11 can allow a lumen to independently move translationally along and rotationally about the longitudinal axis 815. These translational and rotational movements of the lumen are independent of the movements of other lumens of the plurality of lumens 812. The nest 810 is coupled with the carriage in a way to allow the translational and the rotational movements of the lumen it holds.

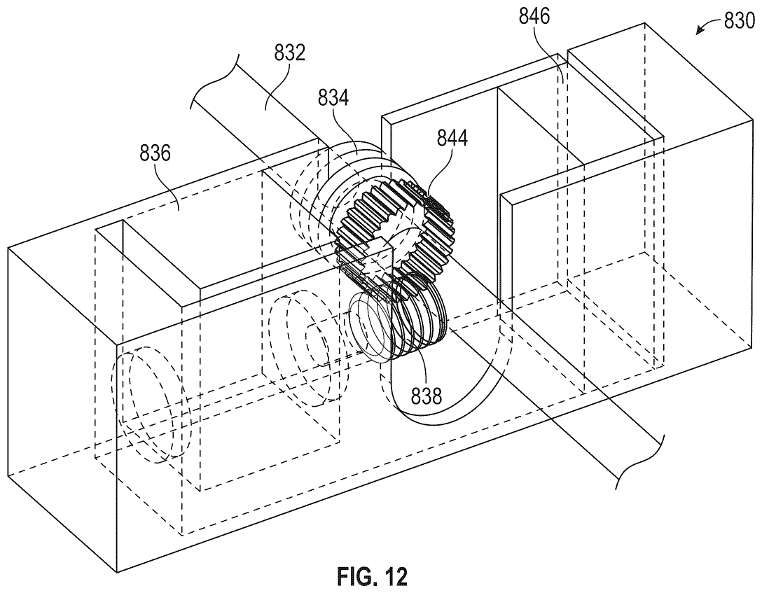

Another embodiment to drive a lumen translationally and rotationally using two motors is illustrated in FIG. 12. A carriage 830 is coupled with a gear for translational movement as described above with respect to FIGS. 2, 3, 4, 7, and 9. As shown in FIG. 12, a motor 836 is coupled with the carriage 830. The motor 836 has a worm gear 838 attached thereon to engage a gear 844, which is attached to a lumen 832. The lumen 832 may be one of a plurality of lumens nested together. The lumen 832 is rotatably coupled with the carriage 830 through an adapter 834, which may be retained by a mechanism (not shown) in the correct gear meshing location. The carriage 830 holds a load cell 846 for measuring a force related to the lumen 832. As can be seen in FIG. 12, when the motor 836 is actuated, it drives the worm gear 838 causing the gear 844 and thus the lumen 832 to rotate. As such, the implementation shown in FIG. 12 can cause translational and rotational movements to the lumen 832. When the movements are stopped, the rotational position of the lumen 832 can be locked by the retaining mechanism (not shown).

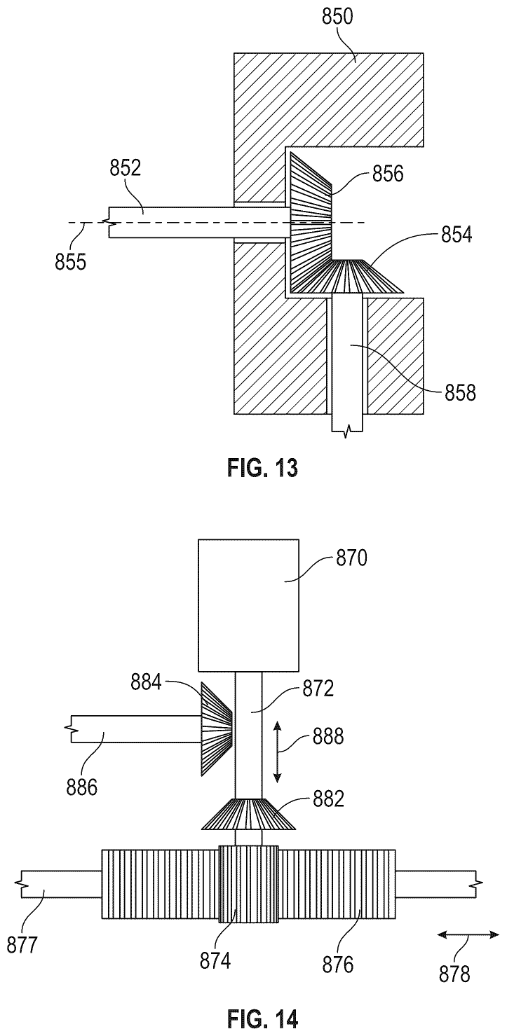

The implementations of FIGS. 10-12 require two motors 816, 824 to realize translational and rotational movements to a lumen. However, in some implementations, it is possible to reduce the number of motors to one to reduce cost and increase compactness of the electronically controlled actuation system and to simplify operation. FIG. 13 schematically illustrates such an implementation. A lumen 852 is coupled with a nest 850 in a way to allow the lumen 852 to translate with the nest 850 along a longitudinal axis 855, when the nest 850 is translationally moved by a motor (not shown), such as described above with respect to FIGS. 2, 3, 4, 7, and 9. The coupling of the lumen 852 with the nest 850 also allows the lumen 852 to rotate about the longitudinal axis 855. By a “clutching” action, the same motor can be engaged with a shaft 858, which is attached to a bevel gear 854. The engagement of bevel gears 854, 856 can allow the shaft 858 to transfer rotational movement to the bevel gear 856, and thus the lumen 852.

An implementation of the bevel gears arrangement illustrated in FIG. 13 is schematically illustrated in FIG. 14. A motor 870 is attached to a pinion gear 874 and a bevel gear 882 through a shaft 872. The pinion gear 874 is engaged with a rack 876 which can be attached to a lumen 877, as described above in FIGS. 10-11. In other embodiments, the attached lumen may also be arranged in parallel to the rack 876. When the motor 870 is actuated, the rotation of the shaft 872 causes the pinion gear 874 to translationally move the rack 876 in a direction 878. This translational movement of the rack 876 can cause the attached lumen 877 to move accordingly. When rotational movement of the lumen is needed, the assembly of the motor 870, the pinion gear 874, and the bevel gear 882 can be moved up, for example, by clutching, so that the bevel gear 882 is engaged with another bevel gear 884, which is attached to a shaft 886. Meanwhile, the pinion gear 874 can be disengaged with the rack 876. In this case, actuating the motor 870 can cause the bevel gears 882, 884 to transfer rotational movement of the shaft 872 to rotational movement of the shaft 886. The rotational movement of the shaft 886 can cause the lumen 877 to rotate (e.g., by gear or belt/pulley mechanism).

In some embodiments, two or more lumens (e.g., two, three, four, five, or all of the lumens) can be controlled to move translationally and/or rotationally simultaneously and independently. This makes it easy to perform the procedures requiring two lumens to be moved at the same time. Also, moving lumens in opposite directions at the same time is difficult to implement in a traditional handle, but becomes easy in the disclosed embodiments. This movement of two lumens in opposite directions may be beneficial for eliminating unwanted instability in the distal end of the catheter system (e.g., drift). Another item that becomes easier to implement with the setups of FIGS. 10-14 is independent clocking control of lumens, because each of the lumens can rotate independently and be locked when the rotational movement is stopped.

In accordance with several embodiments, an independent accessory motor is included (e.g., operably and mechanically coupled) to the actuator or catheter system 100, 200. The independent accessory motor may be mounted in a stationary position or may be translatable, rotatable, and/or otherwise movable along the actuator platform or catheter system 100, 200, depending on the use case. In some implementations, an adapter is operably and/or mechanically attached or otherwise coupled to the motor that allows the motor to interface with different catheter features such as spooling/translating of a suture or wire, torquing a member, or pinning a member. The actuator or catheter system 100, 200 may include a clamp or other mechanical feature for pinning or attaching (e.g., guidewire pinning).

In accordance with several embodiments, the actuator platform or catheter system 100, 200 may include an internal pump (e.g., peristaltic pump) configured and adapted for flushing and/or aspirating one or multiple lumens of the catheter system. In some embodiments, the pump is configured and adapted for expansion of one or more balloons associated with the catheter system. One or more ports of the pump may be accessible to facilitate changing from saline, contrast, or aspiration as desired or required.

In accordance with several embodiments, a sheath holder is provided to fixate or hold a sheath of the introducer or catheter system at a desired area prior to a patient access point as the system 100, 200 is performing its delivery steps. The sheath holder may be used to prevent the sheath from translating when the catheter (e.g., one or more internal lumens of the catheter system 200) is translated.

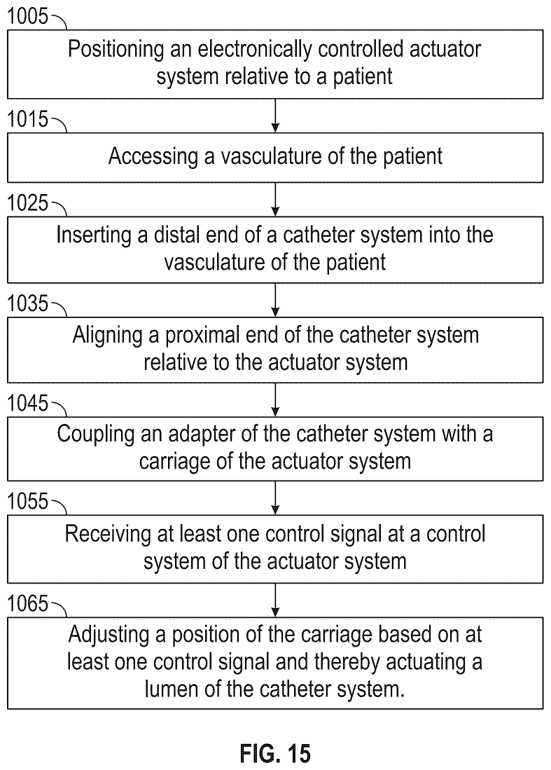

FIG. 15 illustrates a method of controlling a medical system including an electronically controlled actuator system like the system 100 and a catheter system like catheter system 200. The method can be utilized with various different types of catheter systems and to complete various types of medical procedures. At step 1005, the method can include positioning an electronically controlled actuator system of the medical system relative to a patient. The electronically controlled actuator system can include a base and a frame extending along a longitudinal axis. A carriage or a plurality of carriages can be mounted with the frame and movable along the longitudinal axis with respect to the frame. A carriage drive system can adjust positions of the carriages along the longitudinal axis to an initial configuration.

At step 1015, an incision can be made to access a vasculature of the patient. In one example, a transfemoral approach to the heart can be used.

At step 1025, a distal end of a catheter system can be advanced into the vasculature of the patient. The distal end can be advanced into or adjacent to an anatomical region of the patient for proceeding with a medical procedure.

At step 1035, a proximal end of the catheter system can be aligned relative to the electronically controlled actuator system. Optionally, a shell of the handle can be removed and/or adapters can be attached with one or more lumens of the catheter system at the proximal end.

At step 1045, an adapter or a plurality of adapters of the catheter system can be nested with or otherwise coupled to the carriage or carriages. The adapters can be received within slots of the carriages.

At step 1055, a carriage drive system can receive at least one control signal. The control signal can be based on a user input at a user interface. The control signal can be based on a force feedback system of the actuator system (e.g., comparing a force signal measured by a load cell and a permitted force limit of the lumen). The control signal can also be based on an automated procedure. The automated procedure can be based on analysis of one or more images relating to the positions of the catheter system within the anatomical region of the patient.

At step 1065, the actuator system can adjust a position of the carriage or carriages along the longitudinal axis of the frame. The adjustment can be based on at least one control signal. The adjustment can actuate one or more lumens of the catheter system in furtherance of a medical procedure.

In some embodiments, particular anatomical or procedural dimensions, measurements, and/or maneuvers for a particular patient based on preparatory screening or advance planning may be pre-populated and input into the actuator or control system prior to a procedure and the electronically controlled actuator system 100 may be configured to automatically perform initial advancement and positioning maneuvers (e.g., primary and secondary flex maneuvers, depth and/or height translation maneuvers) of one or more lumens of the catheter system (e.g., for replacement heart valve delivery systems, the initial advancement and positioning maneuvers may get the replacement heart valve in a desired general location within the heart). A clinical professional could then perform fine tuning advancement and positioning maneuvers.

### Certain Terminology

Terms of orientation used herein, such as “top,” “bottom,” “proximal,” “distal,” “longitudinal,” “lateral,” and “end,” are used in the context of the illustrated example. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular,” “cylindrical,” “semi-circular,” or “semi-cylindrical” or any related or similar terms, are not required to conform strictly to the mathematical definitions of circles or cylinders or other structures, but can encompass structures that are reasonably close approximations.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

The terms “approximately,” “about,” and “substantially” as used herein represent an amount close to the stated amount that still performs a desired function or achieves a desired result. For example, in some examples, as the context may dictate, the terms “approximately,” “about,” and “substantially,” may refer to an amount that is within less than or equal to 10% of the stated amount. The term “generally” as used herein represents a value, amount, or characteristic that predominantly includes or tends toward a particular value, amount, or characteristic. As an example, in certain examples, as the context may dictate, the term “generally parallel” can refer to something that departs from exactly parallel by less than or equal to 20 degrees. All ranges are inclusive of endpoints.

### SUMMARY

Several illustrative examples of electronically controlled actuators have been disclosed. Although this disclosure has been described in terms of certain illustrative examples and uses, other examples and other uses, including examples and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various examples. All possible combinations and subcombinations of elements and components described herein are intended to be included in this disclosure. No single feature or group of features is necessary or indispensable.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one example in this disclosure can be combined or used with (or instead of) any other portion of any of the steps, processes, structures, and/or devices disclosed or illustrated in a different example or flowchart. The examples described herein are not intended to be discrete and separate from each other. Combinations, variations, and some implementations of the disclosed features are within the scope of this disclosure.

While operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some implementations. Also, the separation of various components in the implementations described above should not be understood as requiring such separation in all implementations, and it should be understood that the described components and systems can generally be integrated together in a single product or packaged into multiple products. Additionally, some implementations are within the scope of this disclosure.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures are drawn and/or shown to scale, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, any methods described herein may be practiced using any device suitable for performing the recited steps.

For purposes of summarizing the disclosure, certain aspects, advantages and features of the inventions have been described herein. Not all, or any such advantages are necessarily achieved in accordance with any particular example of the inventions disclosed herein. No aspects of this disclosure are essential or indispensable. In many examples, the devices, systems, and methods may be configured differently than illustrated in the figures or description herein. For example, various functionalities provided by the illustrated modules can be combined, rearranged, added, or deleted. In some implementations, additional or different processors or modules may perform some or all of the functionalities described with reference to the examples described and illustrated in the figures. Many implementation variations are possible. Any of the features, structures, steps, or processes disclosed in this specification can be included in any example.

In summary, various examples of electronically controlled actuator and related methods have been disclosed. This disclosure extends beyond the specifically disclosed examples to other alternative examples and/or other uses of the examples, as well as to certain modifications and equivalents thereof. Moreover, this disclosure expressly contemplates that various features and aspects of the disclosed examples can be combined with, or substituted for, one another. Accordingly, the scope of this disclosure should not be limited by the particular disclosed examples described above, but should be determined only by a fair reading of the claims.

What is claimed is:

1. A medical system, comprising:
an electronically controlled actuator system, comprising:
a frame extending along a longitudinal axis;
a plurality of carriages mounted with the frame and movable along the longitudinal axis with respect to the frame; and
a carriage drive system configured to adjust a position of each of the plurality of carriages along the longitudinal axis based on at least one control signal; and
a catheter system for providing access within a body cavity, comprising:
a distal end;
a proximal end;
a plurality of lumens extending between the distal end and the proximal end; and
a plurality of adapters, each of the plurality of adapters attached to a lumen of the plurality of lumens;
wherein each of the plurality of adapters is configured to be coupled to a corresponding carriage of the plurality of carriages such that adjusting the position of the corresponding carriage along the longitudinal axis actuates the attached lumen.

2. The medical system of claim 1, wherein each of the plurality of carriages is independently movable along the longitudinal axis.

3. The medical system of claim 2, wherein each of the plurality of carriages includes a nest coupled therein, each nest being configured to rotate about the longitudinal axis.

4. The medical system of claim 1, further comprising:
a control system, comprising:
at least one computer-readable memory having stored thereon executable instructions;
one or more processors in communication with the at least one computer-readable memory and configured to execute the instructions to cause the control system to at least:
receive an input signal; and
generate the at least one control signal based on the input signal.

5. The medical system of claim 4, further comprising:
a user interface for providing the input signal.

6. The medical system of claim 5, further comprising:
a load cell configured to measure a force applied to each lumen of the plurality of lumens, wherein the user interface is configured to display the force relative to a permitted force limit associated with the lumen.

7. The medical system of claim 1, wherein the at least one control signal is configured to adjust the positions of at least two of the plurality of carriages along the longitudinal axis as a first group.

8. The medical system of claim 1, wherein each of the plurality of carriages includes a receiving slot configured to receive one of the plurality of adapters therein.

9. The medical system of claim 1, wherein the frame is rotatable on a rotation axis aligned parallel or generally along the longitudinal axis.

10. The medical system of claim 1, wherein the plurality of lumens are attached with the plurality of adapters by respective guidewires.

11. The medical system of claim 1, further comprising a base or table including an upper surface configured to support the frame, and wherein the plurality of lumens of the catheter system include an outer sheath assembly, a mid-shaft assembly, a rail assembly, and an inner shaft assembly.

12. The medical system of claim 1, wherein the frame comprises a pair of rails extending parallel with the longitudinal axis and the plurality of carriages are mounted on the pair of rails.

13. The medical system of claim 1, wherein the carriage drive system comprises a plurality of motors each provided for a respective one of the plurality of carriages.

14. The medical system of claim 1, wherein each of the plurality of carriages includes a limit switch or proximity switch for homing the carriages relative to the frame.

15. The medical system of claim 1, wherein the at least one control signal is further based on an autonomous or semi-autonomous control algorithm.

16. A method of controlling a medical system, comprising:
positioning an electronically controlled actuator system of the medical system relative to a patient, the electronically controlled actuator system comprising:
a frame extending along a longitudinal axis;
a carriage mounted with the frame and movable along the longitudinal axis with respect to the frame; and
a carriage drive system configured to adjust a position of the carriage along the longitudinal axis;
making an incision to access a vasculature of the patient;
inserting a distal end of a catheter system into the vasculature of the patient through the incision and advancing the distal end through the vasculature into or adjacent to an anatomical region of the patient;
aligning a proximal end of the catheter system relative to the electronically controlled actuator system;
coupling an adapter of the catheter system with the carriage;
receiving at least one control signal at a control system of the electronically controlled actuator system; and
adjusting a position of the carriage along the longitudinal axis based on at least one control signal and thereby actuating a lumen of the catheter system.

17. The method of claim 16, further comprising receiving an input signal from a user interface and generating the at least one control signal based on the input signal.

18. The method of claim 16, further comprising:
receiving a force signal from a load cell and generating the at least one control signal based on the force signal.

19. The method of claim 18, further comprising:
determining an available range of motion of the lumen based on the force signal measured by the load cell and a permitted force limit of the lumen.

20. The method of claim 16, further comprising:
adjusting the position of the carriage to an initial, pre-programmed location before coupling the adapter of the catheter system with the carriage.
